SEQUENCE LISTING (13 sequences)

SEQ ID NO: 1 - DNA, 18 bp, Homo sapiens
acggacctggatataggg

SEQ ID NO: 2 - DNA, 18 bp, Homo sapiens
tgggtaggtccccattgg

SEQ ID NO: 3 - DNA, 18 bp, Homo sapiens
gagtcaacggatttggtc

SEQ ID NO: 4 - DNA, 18 bp, Homo sapiens
ccatgccagtgagctttc

SEQ ID NO: 5 - DNA, 45 bp, Artificial Sequence
  universal primer
ctaatacgactcactatagggcaagcagtggtatcaacgcagagt

SEQ ID NO: 6 - DNA, 25 bp, Homo sapiens
ccacatatggcttttcatggactcg

SEQ ID NO: 7 - DNA, 25 bp, Homo sapiens
cttccgctcctgtgcctgcaaacta

SEQ ID NO: 8 - DNA, 21 bp, Homo sapiens
tacgatcggcctcctgggatg

SEQ ID NO: 9 - DNA, 25 bp, Homo sapiens
cttccgctcctgtgcctgcaaacta

SEQ ID NO: 10 - DNA, 16 bp, Homo sapiens
atcaccgcggttcctg

SEQ ID NO: 11 - DNA, 21 bp, Homo sapiens
acggaaggctaagcaaggctg

SEQ ID NO: 12 - DNA, 40 bp, Homo sapiens
ggggcagatccaggttcaggaaactgggcatctctgtggc

SEQ ID NO: 13 - DNA, 40 bp, Homo sapiens
ggggcagatccaggttcaggatggacatggtagagaatgc

(19) United States
(12) Patent Application Publication
(10) Pub. No.: US 2012/0164642 A1
(43) Pub. Date: Jun. 28, 2012

(54) METHODS AND MATERIALS FOR IDENTIFYING NODULAR FASCIITIS

(76) Inventors: Michele R. Erickson-Johnson, Rochester, MN (US); Andre M. Oliveira, Rochester, MN (US); Margaret Chou, Bala Cynwyd, PA (US)

(21) Appl. No.: 13/335,438

(22) Filed: Dec. 22, 2011

Related U.S. Application Data
(60) Provisional application No. 61/425,988, filed on Dec. 22, 2010.

(51) Int. Cl. C12Q 1/68

(52) U.S. Cl. 435/6.11

(57) ABSTRACT

This document provides methods and materials involved in detecting gene rearrangements indicative of nodular fasciitis. For example, methods and materials for determining if a mesenchymal neoplasm is nodular fasciitis based at least in part on the detection of a gene rearrangement (e.g. a USP6 or MYH9 rearrangement) are provided.

### CROSS-REFERENCE TO RELATED APPLICATIONS

This application claims the benefit of U.S. Provisional Application Ser. No. 61/425,988, filed Dec. 22, 2010. The disclosure of the prior application is considered part of (and is incorporated by reference in) the disclosure of this application.

### BACKGROUND

1. Technical Field

This document relates to methods and materials involved in detecting gene rearrangements indicative of nodular fasciitis. For example, this document provides methods and materials for determining if a mesenchymal neoplasm is nodular fasciitis based at least in part on the detection of a gene rearrangement (e.g. a USP6 or MYH9 rearrangement).

2. Background Information

Nodular fasciitis (also known as pseudosarcomatous fasciitis) is a relatively common dermal mesenchymal lesion that occurs in the upper extremities, trunk, head, and neck of children and young adults and predominantly affects younger individuals. Due to characteristic rapid growth and rich cellular and mitotic activity, nodular fasciitis may be misdiagnosed as a sarcoma. The deceptive histologic appearance commonly results in erroneous diagnosis and unnecessary radical overtreatment.

### SUMMARY

This document provides methods and materials related to identifying or assessing mammals (e.g., humans) for the presence or absence of cells having one or more gene rearrangements indicative of nodular fasciitis. For example, this document provides methods and materials that can be used to determine whether or not a mammal contains cells having USP6 or MYH9 gene rearrangements. As described herein, the presence of cells having a USP6 rearrangement, an MYH9 rearrangement, or an MYH9-USP6 fusion gene can indicate that a mammal with a mesenchymal neoplasm has nodular fasciitis, as opposed to, for example, a sarcoma. Having the ability to identify a mammal (e.g., a human) as having nodular fasciitis as opposed to a sarcoma can allow clinicians and patients to proceed with appropriate treatment for nodular fasciitis while avoiding unnecessary treatments aimed at sarcomas.

In general, one aspect of this document features a method of distinguishing nodular fasciitis from cancer in a human with a mesenchymal neoplasm. The method comprises, or consists essentially of, determining whether or not a cell from the mesenchymal neoplasm of the human contains a USP6 rearrangement or a MYH9 rearrangement, wherein the presence of a USP6 or MYH9 gene rearrangement in the mesenchymal neoplasm cell indicates that the mammal has nodular fasciitis. The cancer can be sarcoma. The gene rearrangement can be a translocation between USP6 and MYH9. The translocation can result in the coding region of USP6 being under the control of a MYH9 promoter. The determining step can comprises in situ hybridization. For example, the determining step can be fluorescent in situ hybridization (FISH), chromogenic in situ hybridization (CISH), or silver in situ hybridization (SISH). The determining step can comprise in situ hybridization with a USP6 probe set. The determining step can comprise in situ hybridization with a MYH9 probe set.

In another aspect, this document features an article of manufacture comprising, or consisting essentially of, a container comprising, or consisting essentially of, a probe having the ability to hybridize to human chromosome 17 sequences and a probe set comprising, or consisting essentially of, probes having the ability to hybridize to human chromosome 22 sequences, wherein the probe and the probe set have the ability to detect a t(17;22). The probe can comprise, or consist essentially of, a USP6 probe. The probe can comprise, or consist essentially of, a MYH9 probe. The probe can comprise a fluorescent, chromogenic, or silver label. The probes of the probe set can comprise a different fluorescent label.

In another aspect, this document features a method for detecting the presence or absence of an USP6-MYH9 gene rearrangement, wherein the method comprises, or consists essentially of, hybridizing to the nucleic acid of a cell a probe set comprising at least one isolated nucleic acid molecule having the ability to hybridize to a USP6 nucleotide sequence and at least one isolated nucleic acid molecule having the ability to hybridize to a MYH9 nucleotide sequence to form a hybridization pattern, and determining whether or not the hybridization pattern is a pattern of gene rearrangement. The isolated nucleic acid molecules can be labeled. The at least one isolated nucleic acid molecule having the ability to hybridize to a USP6 nucleotide sequence can comprise a label that is different from the label of the isolated nucleic molecule having the ability to hybridize to a MYH9 nucleotide sequence.

In another aspect, this document features a method of identifying a mammal having a mesenchymal neoplasm as having nodular fasciitis. The method comprises, or consists essentially of, (a) determining whether or not a cell from the mesenchymal neoplasm contains a USP6 or MYH9 rearrangement, and (b) classifying the mammal as having nodular fasciitis if the cell contains the rearrangement. The USP6 or MYH9 rearrangement can be a translocation between USP6 and MYH9. The translocation can result in the coding region of USP6 being under the control of a MYH9 promoter. The determining step can comprise performing in situ hybridization. The determining step can comprise performing in situ hybridization with a USP6 probe set. The determining step can comprise performing in situ hybridization with a MYH9 probe set. The determining step can comprise performing an assay to detect increased expression of a USP6 nucleic acid, wherein the increased expression indicates the presence of the rearrangement. The mammal can be a human.

In another aspect, this document features a method of identifying a mammal having a mesenchymal neoplasm as having nodular fasciitis. The method comprises, or consists essentially of, (a) detecting the presence of a USP6 or MYH9 rearrangement in a cell from the mesenchymal neoplasm, and (b) classifying the mammal as having nodular fasciitis based at least in part on the presence of the rearrangement. The USP6 or MYH9 rearrangement can be a translocation between USP6 and MYH9. The translocation can result in the coding region of USP6 being under the control of a MYH9 promoter. The determining step can comprise performing in situ hybridization. The determining step can comprise performing in situ hybridization with a USP6 probe set. The determining step can comprise performing in situ hybridization with a MYH9 probe set. The detecting step can comprise detecting increased expression of a USP6 nucleic acid, wherein the increased expression indicates the presence of the rearrangement. The mammal can be a human.

In another aspect, this document features a probe set comprising, or consisting essentially of, at least one isolated nucleic acid molecule having the ability to hybridize to a USP6 nucleotide sequence and at least one isolated nucleic acid molecule having the ability to hybridize to a MYH9 nucleotide sequence. The isolated nucleic acid molecules can be labeled. The at least one isolated nucleic acid molecule having the ability to hybridize to a USP6 nucleotide sequence can comprise a label that is different from the label of the isolated nucleic molecule having the ability to hybridize to a MYH9 nucleotide sequence. The labels can be fluorescent, chromogenic, or silver.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a schematic of bacterial artificial chromosome (BAC) clones in relationship to USP6 at chromosome band 17p13 and in relationship to MYH9 at chromosomal band 22q12. Dashed vertical lines indicate the consensus genomic breakpoint regions, as determined by fluorescent in situ hybridization. The “X” indicates that the MYH9 promoter is swapped for USP6's promoter and the illustration below represents the new fusion product (der=derivative). Red fluorescently-labeled BAC clone probes are shown as double bars, and green fluorescently-labeled BAC clone probes are shown as triple bars. cen=centromeric side of the andchromosome, and pter=telomeric side of the chromosome. FIG. 1B is a diagram showing the predicted MYH9-USP6 fusion transcripts. Type 1 has MYH9 noncoding exon 1 fused to part of USP6 noncoding exon 1 (Genbank Accession No. 1385057), and Type 2 has MYH9 noncoding exon 1 fused with USP6 coding exon 2 (Genbank Accession no. 1394854). White areas indicate noncoding exons, while the light gray areas indicate coding sequences. Protein domains are represented in gray and include TBC (TBC/GAP GTPase domain) and UBP (ubiquitin protease domain). The numbers indicate exon numbers for MYH9 and USP6. FIG. 1C includes nucleic acid sequences of the splicing junctions in type 1 and type 2 MYH9-USP6 fusion genes. The USP6 ATG initiation codon is underlined.

FIG. 2 contains histology images of (A) human nodular fasciitis (HE×100) and (B) mouse tumor (HE×100) induced by USP6-transfected preosteoblasts. FIG. 2C is a photograph of a semi-quantitative RT-PCR assay. Lane 1 contains parental MC3T3. Lane 2 contains an MC3T3/USP6 long isoform (−1.2 Kb). Lane 3 contains MC3T3/USP6, short isoform (−1.1 Kb). Lane 4 contains nucleic acid from a patient with nodular fasciitis (patient 1). Lane 5 contains nucleic acid from a patient with nodular fasciitis (patient 2). Lane 6 contains dH₂O. Lane 7 contains GAPDH nucleic acid (600 bp) from a patient with nodular fasciitis (patient 1). Lane 8 contains GAPDH nucleic acid (600 bp) from a patient with nodular fasciitis (patient 2). Lane M contains a 1 Kb marker ladder.

FIG. 3A is a photograph of a FISH experiment showing rearrangement of the USP6 locus. FIG. 3B is a photograph of a 5′ RACE PCR assay showing products separated on a gel. Lane 1 contains dH₂O, lane 2 contains an MYH9-USP6 fusion (˜213 bp) using UPM+USP6_{—}3644 (arrow), lane 3 contains a UPM+1746 USP6 primer, and lane M=a 25 bp marker ladder. FIG. 3C contains a sequencing chromatograph of Type 1 MYH9-USP6 seen in a nodular fasciitis patient (patient 1) by both RACE and RT-PCR.

FIG. 4A is a photograph of a FISH experiment showing rearrangements of the MYH9 locus. FIG. 4B is a photograph of a bring together FISH approach (D-FISH) with 5′ USP6 labeled in spectrum green (appears as gray dots inside cells) and 3′ MYH9 labeled in spectrum orange (appears as intense white dots inside cells). FIG. 4C (top panel) contains RT-PCR results using nucleic acid from four different patients with nodular fasciitis (lanes 1-4), water, or nucleic acid from patients with the indicated condition. The type 1 MYH9-USP6 (˜173 bp) fusion was only seen in patients with nodular fasciitis and not in other tumors studied. ARMS is an abbreviation for alveolar rhabdomyosarcoma, and DSRCT is an abbreviation for desmoplastic small round cell tumor. Schwannoma produced a nonspecific band that blasted to chromosome 16. The lower panel contains RT-PCR results for PGK nucleic acid (189 bp), a RNA integredity control. PGK is an abbreviation for phosphoglycerate kinase.

### DETAILED DESCRIPTION

This document provides methods and materials related to identifying or assessing mammals (e.g., humans) for the presence or absence of cells having one or more gene rearrangements indicative of nodular fasciitis. For example, this document provides methods and materials that can be used to determine whether or not a mammal contains cells having USP6 or MYH9 gene rearrangements. As described herein, the presence of cells having a USP6 rearrangement, an MYH9 rearrangement, or an MYH9-USP6 fusion gene can indicate that a mammal with a mesenchymal neoplasm has nodular fasciitis. For example, the methods and materials provided herein can be used to determine whether or not a mammal (e.g., a human) contains cells having a USP6 rearrangement, an MYH9 rearrangement, or an MYH9-USP6 fusion gene, and the presence of such cells can indicate that that mammal has nodular fasciitis as opposed to a sarcoma.

Any appropriate method can be used to assess a mammal for the presence or absence of cells having a USP6 rearrangement, MYH9 rearrangement, or MYH9-USP6 fusion gene. For example, in situ hybridization assays (e.g., fluorescent in situ hybridization (FISH), chromogenic in situ hybridization (CISH), or silver in situ hybridization (SISH)) can be used to determine whether or not a mammal contains cells having a USP6 rearrangement, an MYH9 rearrangement, or an MYH9-USP6 fusion gene. Other methods that can be used include, without limitation, PCR and end-sequence profiling, RACE PCR, and RT-PCR. If a mammal is determined to contain cells having a USP6 rearrangement, an MYH9 rearrangement, or an MYH9-USP6 gene fusion, then that mammal can be classified as having nodular fasciitis based at least in part on the presence of such cells.

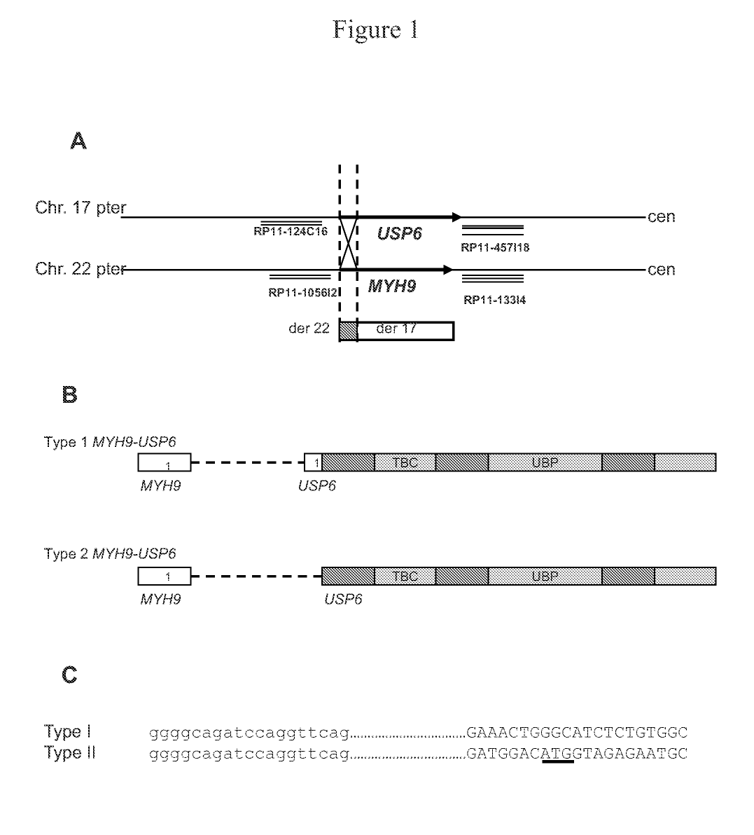

In one embodiment, this document provides a collection of nucleic acid molecules (e.g., probes) having the ability to detect a USP6-MYH9 rearrangement. Such collections can contain at least one (e.g., 2, 3, 4, 5, 10, or more) nucleic acid molecule having the ability to hybridize to a USP6 nucleotide sequence and at least one (e.g., 2, 3, 4, 5, 10, or more) nucleic acid molecule having the ability to hybridize to a MYH9 nucleotide sequence. For example, a collection of isolated nucleic acid molecules provided herein can contain at least one isolated nucleic acid molecule having the ability to hybridize to a USP6 nucleotide sequence and at least one isolated nucleic acid molecule having the ability to hybridize to a MYH9 nucleotide sequence. Examples of isolated nucleic acid molecules having the ability to hybridize to a USP6 nucleotide sequence include, without limitation, RP11-167N20, RP11-910H9, RP11-211L24, RP11-106A7, RP11-198F11, RP11-115H24, RP11-124C16, RP11-457I18, RP11-1140D18, RP11-790C7, RP11-373N8, RP11-80K10, and RP11-960B9. Examples of isolated nucleic acid molecules having the ability to hybridize to a MYH9 nucleotide sequence include, without limitation, RP11-175G10, RP11-347K20, RP11-241E17, RP11-133I4, RP11-1056I22, RP11-643I13, and RP11-846O5. FIG. 1 shows the possible relation of the USP6 and MYH9 probes to genomic breakpoint regions in nodular fasciitis. Collections of isolated nucleic acid molecules having the ability to detect USP6-MYH9 rearrangements can include a vector such as a bacterial artificial chromosome (BAC) or a fosmid. For example, a collection of nucleic acid molecules provided herein can be a collection of BACs containing nucleotide sequences capable of hybridizing to USP6 nucleic acid or MYH9 nucleic acid. Isolated nucleic acid molecules having the ability to detect USP6-MYH9 rearrangements can be any length. In some cases, isolated nucleic acid molecules provided herein (e.g., nucleic acid molecules having the ability to detect USP6-MYH9 rearrangements) can be more than 50 bp in length (e.g., more than 100 bp, 250 bp, 500 bp, 1 kb, 2 kb, 5 kb, 7 kb, 10 kb, 20 kb, 50 kb, 100 kb, or 300 kb). Isolated nucleic acid molecules provided herein can have sequences that overlap with another member of the collection. In some cases, each nucleic acid molecule of a collection can have a sequence that is distinct from the sequences of the other members of the collection. The isolated nucleic acid molecules of a collection provided herein can hybridize to USP6 or MYH9 nucleotide sequences present in either an intron or an exon. Introns and exons to which isolated nucleic acid molecules have the ability to detect USP6-MYH9 rearrangements can hybridize upstream or downstream of the transcription start site or the termination codon of a USP6 or MYH9 nucleotide sequence.

The term “nucleic acid” as used herein can be RNA or DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated nucleic acid” as used herein includes any non-naturally-occurring nucleic acid sequence since such non-naturally-occurring sequences are not found in nature and do not have immediately contiguous sequences in a naturally-occurring genome. An isolated nucleic acid can be, for example, a DNA molecule, provided one of the nucleic acid sequences normally found immediately flanking that DNA molecule in a naturally-occurring genome is removed or absent.

One or more of the isolated nucleic acid molecules provided herein can be labeled (e.g., fluorescently, biotin-labeled, antigen-labeled, or radioactively labeled) and can be used as a probe (e.g., fluorescent in situ hybridization (FISH) probe). In some cases, the collections of isolated nucleic acid molecules provided herein can be dual fusion FISH probe sets (e.g., D-FISH probe sets) that can be used to identify gene rearrangement fusion products (e.g., USP6-MYH9). A D-FISH probe set can have (1) at least one probe that is labeled with a fluorophore (e.g., SpectrumGreen™ or SpectrumOrange™) and that has the ability to hybridize to a nucleotide sequence of one gene (e.g., a USP6 gene), and (2) at least another probe that is labeled with a different fluorophore and that has the ability to hybridize to a nucleotide sequence of a second gene (e.g., a MYH9 gene). Such D-FISH probe sets can be used to determine whether 0, 1, or 2 alleles have rearranged. For example, when hybridized in situ to a target gene, a USP6 D-FISH probe that is labeled with SpectrumOrange™ and a MYH9 D-FISH probe that is labeled with SpectrumGreen™ can produce two green foci and two red foci within the nucleus. Such a pattern or result can be termed 2R2G and can indicate that neither gene locus is rearranged. In another example, the hybridization described above can produce one green focus, one red focus, and two yellow (e.g., red-green fusion) foci. Such a pattern or result can be termed 1R1G2F, or classic D-FISH signal pattern, and can indicate that one allele of each locus is intact and the other USP6 allele has recombined with a MYH9 allele.

In some cases, a collection of isolated nucleic acid molecules provided herein can be a collection of break apart FISH probes (e.g., BAP probes) that can be used to determine the breakpoint in a rearranged gene (e.g., USP6 or MYH9). In some cases, a BAP probe set can include more than one isolated nucleic acid molecule (e.g., more than 2, 3, 4, 5, or more nucleic acid molecules). For example, a USP6 BAP probe set can contain one or more of the following nucleic acid molecules: RP11-167N20, RP11-910H9, RP11-211L24, RP11-106A7, RP11-198F11, RP11-115H24, RP11-124C16, RP11-457I18, RP11-1140D18, RP11-790C7, RP11-373N8, RP11-80K10, and RP11-960B9 nucleic acid molecules. A MYH9 BAP probe set can contain one or more of the following nucleic acid molecules: RP11-175G10, RP11-347K20, RP11-241E17, RP11-133I4, RP11-1056I22, RP11-643I13, and RP11-846O5 nucleic acid molecules. BAP probe sets can contain at least one isolated nucleic acid molecule that is labeled with a fluorophore and has the ability to hybridize to the centromeric region of a gene and at least one other isolated nucleic acid molecule that is labeled with a different fluorophore and has the ability to hybridize to the telomeric region of the gene. Such BAP probe sets can be hybridized to a target gene locus using in situ hybridization to visualize the breakpoint in a rearranged gene. For example, when hybridized in situ to the target gene, an USP6 BAP probe set containing (1) a probe that has the ability to hybridize to the centromeric end of the gene and that is labeled with SpectrumOrange™, and (2) a probe that has the ability to hybridize to the telomeric end of the gene and is labeled with SpectrumGreen™, can produce one red focus, one green focus, and one yellow (e.g., red-green fusion) focus. Such a pattern or result can be termed 1R1G1F and can indicate that one USP6 allele is intact and the second allele has rearranged at a breakpoint that is between the portion of the probe labeled with SpectrumGreen™ and the portion of the probe labeled with SpectrumOrange™

SpectrumOrange™-labeled nucleic acid can be used to generate a signal that can be referred to as red (“R”). SpectrumGreen™-labeled nucleic acid can be used to generate a signal that can be referred to as green (“G”). SpectrumAqua™-labeled nucleic acid can be used to generate a signal that can be referred to as aqua (“AQ”). Proximal signals from SpectrumOrange™-labeled nucleic acid and SpectrumGreen™-labeled nucleic acid can combine to form a fusion (“F”) signal. Fusion signals can be distinguishable from other signals as adjacent red and green signals or fusion signals can appear as a combined red-green signal (e.g., yellow). It will be understood that the fluorophores used herein can be substituted with alternative sets of distinguishable fluorophores.

This document also provides kits containing a probe set having two or more nucleic acid probes that are at least 10,000 nucleotides in length (e.g., at least 20,000 nucleotides in length; at least 50,000 nucleotides in length; at least 75,000 nucleotides in length; at least 100,000 nucleotides in length; or at least 150,000 nucleotides in length) and the have the ability to hybridize to a sequence from chromosome 17 and a sequence from chromosome 22. Such probes that hybridize to a sequence from chromosome 17 can be a BAC clone designated as RP11-167N20, RP11-910H9, RP11-211L24, RP11-106A7, RP11-198F11, RP11-115H24, RP11-124C16, RP11-457I18, RP11-1140D18, RP11-790C7, RP11-373N8, RP11-80K10, or RP11-960B9. Such probes that hybridize to chromosome 22 can be a BAC clone designated as RP11-175G10, RP11-347K20, RP11-241E17, RP11-133I4, RP11-1056I22, RP11-643I13, or RP11-84605. The kits provided herein can contain one or more probes having the ability to hybridize to a sequence from chromosome 17 and chromosome 22. Such kits can be used to detect a translocation between chromosome 17 and chromosome 22.

In situ hybridization using the nucleic acids provided herein can be performed using any appropriate technique, such as interphase, metaphase, or fiber FISH. Such techniques can be performed on cells of fresh-fixed or paraffin-embedded tissue samples. Cells from any mesenchymal neoplasm biopsy sample can be used. Microscopy can then be used to detect the presence or absence of a gene rearrangement. In the case of a D-FISH probe set, a gene rearrangement is typically identified by the presence of one or more fusion signals. For example, an MYH9 (chromosome 22) telomeric side probe can be labeled with Spectrum Orange, and a USP6 (chromosome 17) centromeric side probe can be labeled with Spectrum Green. In such cases, a 2R2G pattern can indicate a normal cell, and a 1R1G1Y pattern can indicate a fusion. In the case of a BAP probe set, a gene rearrangement is typically identified by the presence of one or more non-fusion signals (e.g., a red or a green signal). The pattern and size of a signal can be used to estimate the location of a breakpoint. For example, a large non-fusion red signal, a non-fusion green signal, and a single fusion signal that are produced when using a BAP probe set containing (1) a probe that has the ability to hybridize to the centromeric end of a gene and that is labeled with SpectrumOrange™, and (2) a probe that has the ability to hybridize to the telomeric end of the gene and is labeled with SpectrumGreen™, can indicate that the break point in that gene is more telomeric than if a small non-fusion red signal, a non-fusion green signal, and a single fusion signal were produced.

In some cases, the methods provided herein can be used to determine whether a mammal has nodular fasciitis. Methods for determining whether a mammal has nodular fasciitis can include identifying a mammal suspected of having nodular fasciitis and determining from a tissue sample from that mammal the percentage of cells having a USP6 or MYH9 gene rearrangements (e.g., USP6-MYH9 fusion gene). The percentage of cells having a USP6-MYH9 rearrangement can be determined by hybridizing nucleic acid from a tissue sample of the mammal with a BAP probe for USP6 or MYH9, or a D-FISH probe for USP6-MYH9, and calculating the percentage of cells having a USP6-MYH9 rearrangement. In some cases, a mammal having a mesenchymal neoplasm may be suspected of having sarcoma, and the detection of USP6 or MYH9 rearrangements can be used to distinguish nodular fasciitis from sarcoma. A mammal exhibiting a mesenchymal neoplasm and that is found to have cells having a USP6-MYH9 rearrangement can be classified as having nodular fasciitis based at least in part on the presence of such cells.

In some cases, the presence of USP6 transcriptional upregulation can be used to determine whether a mammal has nodular fasciitis. For example, methods such as semi-quantitative PCR and real time PCR can be used to determine if mesenchymal neoplasm cells contain USP6 transcriptional upregulation. The presence of such cells can indicate that the mesenchymal neoplasm is nodular fasciitis as opposed to a tumor such as a sarcoma. Any appropriate nucleic acid probe or primer set can be used to determine if USP6 is transcriptionally upregulated. Examples of such primers include, without limitation, those set forth in SEQ ID NO:1 and SEQ ID NO:2.

The invention will be further described in the following examples, which do not limit the scope of the invention described in the claims.

### EXAMPLES

### Example 1

### Comparison of Mouse Model of USP6 Overexpression with Human Nodular Fasciitis

### Generation of MC3T3 Cell Lines

Stable cell lines expressing USP6 were generated in MC3T3-E1 pre-osteoblasts (obtained from the ATCC) as described elsewhere (Welman et al., Nat. Protoc., 1(2):803-811 (2006)). USP6 expression was placed under the control of a doxycycline (dox)-inducible promoter.

### Xenografts

All mouse procedures were conducted in accordance with guidelines set by IACUC at the University of Pennsylvania and Children's Hospital of Philadelphia. Nude mice (male, 4-8 weeks) were obtained from Jackson Laboratories and fed water containing 1 mg/mL doxycycline (Research Products International, Mt. Prospect, Ill.) supplemented with 5% sucrose for 1-2 weeks prior to xenografting. USP6-expressing MC3T3-E1 cell lines were pre-treated with or without doxycycline (2 μg/mL) for 24 hours. Cells (2.5E6 in 250 μL) were injected subcutaneously into the rear flank. Doxycycline was maintained in the drinking water and animals were monitored every 1-2 days for a period of 2-4 weeks (Ye et al., Oncogene, 29(25):3619-3629 (2010)).

### Correlation of Mouse Model of USP6 Overexpression to Human Nodular Fasciitis

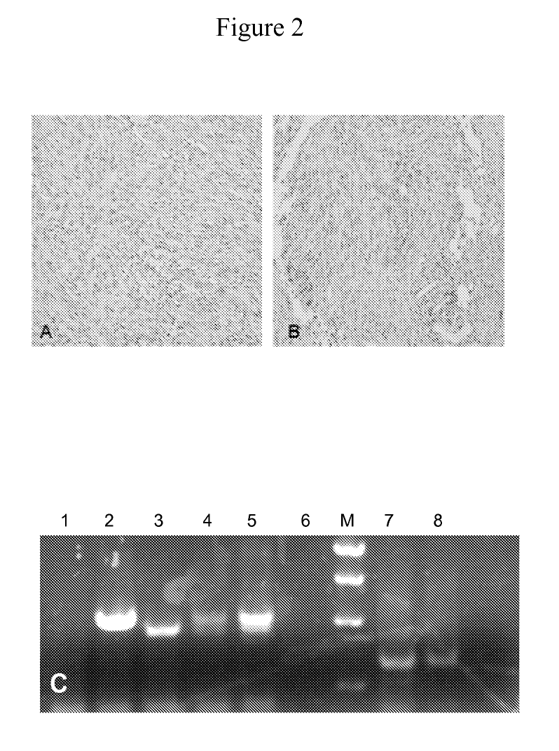

Tumors were paraffin-embedded and histological analysis was performed by the Abramson Cancer Center Pathology Core of the University of Pennsylvania and Mayo Clinic. Histologic sections (4-8 μm) were prepared and subjected to hematoxylin and eosin staining Histologically, xenografts yielded lesions very similar to human nodular fasciitis (FIGS. 2A and 2B). Lesions were characterized by rapid proliferation of spindle cells associated with areas of hemorrhage. All examined lesions lasted for 1-2 weeks and then regressed, closely mimicking the clinico-pathologic features of human nodular fasciitis. This led to the analysis of USP6 mRNA expression in human nodular fasciitis tumors. RNA from two frozen samples was mechanically homogenized using Trizol (Invitrogen, Carlsbad, Calif.) and extracted according to established methods as described elsewhere (Qian et al., Diagn. Mol. Pathol., 14(1):23-28 (2005)). To confirm expression of USP6 mRNA in nodular fasciitis, RT-PCR was performed using the Easy-A One Tube RT-PCR system per manufacturer instructions (Stratagene, Santa Clara, Calif.). RT-PCR was performed using 1.0 μg of RNA and primers for USP6:

| | |
|---|---|
| 5′ ACGGACCTGGATATAGGG | (SEQ ID NO: 1) |
| 5′ TGGGTAGGTCCCCATTGG | (SEQ ID NO: 2) |

and primers for GADPH:

| | |
|---|---|
| 5′ GAGTCAACGGATTTGGTC | (SEQ ID NO: 3) |
| 5′ CCATGCCAGTGAGCTTTC. | (SEQ ID NO: 4) |

High levels of USP6 mRNA expression were found in both human nodular fasciitis tumor samples (FIG. 2C, lanes 4 and 5).

### Example 2

### USP6 Rearrangements in Nodular Fasciitis

### Samples

Forty-five cases of nodular fasciitis were identified at Mayo Clinic, Rochester. All cases exhibited classic histologic features of nodular fasciitis: a subcutaneous cellular spindle cell proliferation with prominent mitotic activity. Negative controls included three schwannomas, three desmoids-type fibromatosis, two dermatofibrosarcoma protuberans, and one of each of the following: ischemic fasciitis, myofibroma, giant cell tumor of tendon sheath, neurofibroma, soft tissue perineurioma, glomangioma, squamous cell carcinoma, synovial sarcoma, desmoplastic small round cell tumor, alveolar rhabdomyosarcoma, and normal thyroid tissue. Aneurysmal bone cysts with USP6 fusion genes were used as positive controls.

### FISH

Bacterial artificial chromosome (BAC) clones flanking USP6 (17p13) and MYH9 (22q13.1) were obtained from Children's Hospital Oakland Research Institute (Oakland, Calif.). USP6 BACs were as described elsewhere (Oliveira et al., Oncogene, 24(21):3419-3426 (2005)). DNA isolation, nick translation, and hybridization were performed as described elsewhere (Sukov et al., Skeletal Radiol., 37(4):321-327 (2008)). Tissues were scored by two independent investigators and considered positive if 10% of 200 cells showed split signals. FISH images were captured using a Leica DM 6000 scope (Leica, Bannockbun, Ill.) with Cytovision software (Genetix, Boston, Mass.).

### Results

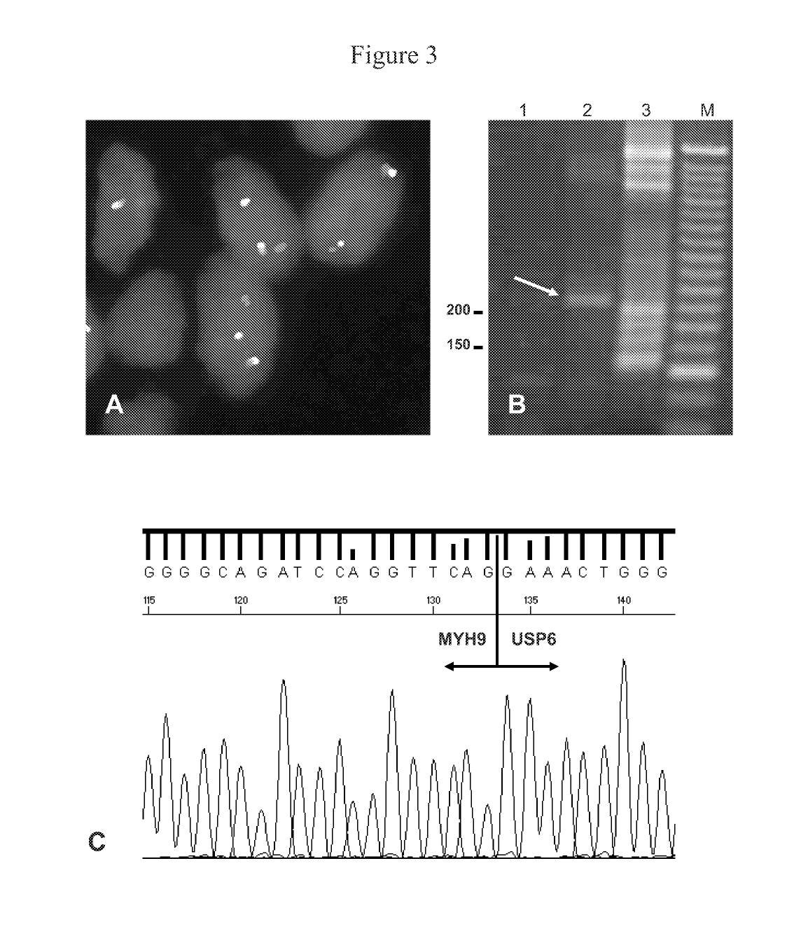

FISH for the USP6 locus in nodular fasciitis samples exhibited a balanced rearrangement of USP6 locus (FIG. 3A). Balanced rearrangements occurred in 41 of 45 (91%) of cases of human nodular fasciitis and not in any of the control tissues and tumors (FIG. 3A). Since the oncogenic mechanism of USP6 in aneurysmal bone cysts is transcriptional upregulation mediated by a fusion to a partner gene with a strong promoter (CDH11 or COL1A1), the involvement of these genes in human nodular fasciitis were excluded by FISH and specific RT-PCR. Based upon the increased USP6 expression levels, rearrangement leads to promoter swapping and transcriptional upregulation of USP6.

### Example 3

### Determination and Confirmation of USP6 Gene Fusion Partner

### Samples

Samples were the same as described above.

### RACE-PCR

RACE-ready cDNA was generated using 1 μg of RNA from nodular fasciitis patient 1 and patient 2 using SMARTer RACE cDNA Amplification kit per manufacturer instructions (Clonetech, Mountain View, Calif.). 5′RACE PCR was performed using touch down PCR (5 cycles of 94° C. 30 sec, 72° C. 3 min; 5 cycles of 94° C. 30 sec, 70° C. 30 sec, 72° C. 3 min; and 25 cycles at 94° C. 30 sec, 68° C. 30 sec and 72° C. 3 min) with the following primers:

universal primer:

(SEQ ID NO: 5)
5′ CTAATACGACTCACTATAGGGCAAGCAGTGGTATCAACGCAGAGT

### USP6 3644:

| | |
|---|---|
| 5′ CCACATATGGCTTTTCATGGACTCG | (SEQ ID NO: 6) |

### USP6 1747:

| | |
|---|---|
| 5′ CTTCCGCTCCTGTGCCTGCAAACTA | (SEQ ID NO: 7) |

Products were visualized on 3% gels and photographed using a DigiDoc-It Imager (UVP, Upland, Calif.). DNA was extracted using Qiaquick get extraction columns per manufacturer instructions (Qiagen, Valencia, Calif.). 5′ RACE on nodular fasciitis tumors was performed to identify a possible novel partner to USP6. Both tumors tested exhibited the fusion of MYH9 exon 1 to USP6 exons 1 and 2 (FIGS. 3B and 3C).

### FISH for MYH9

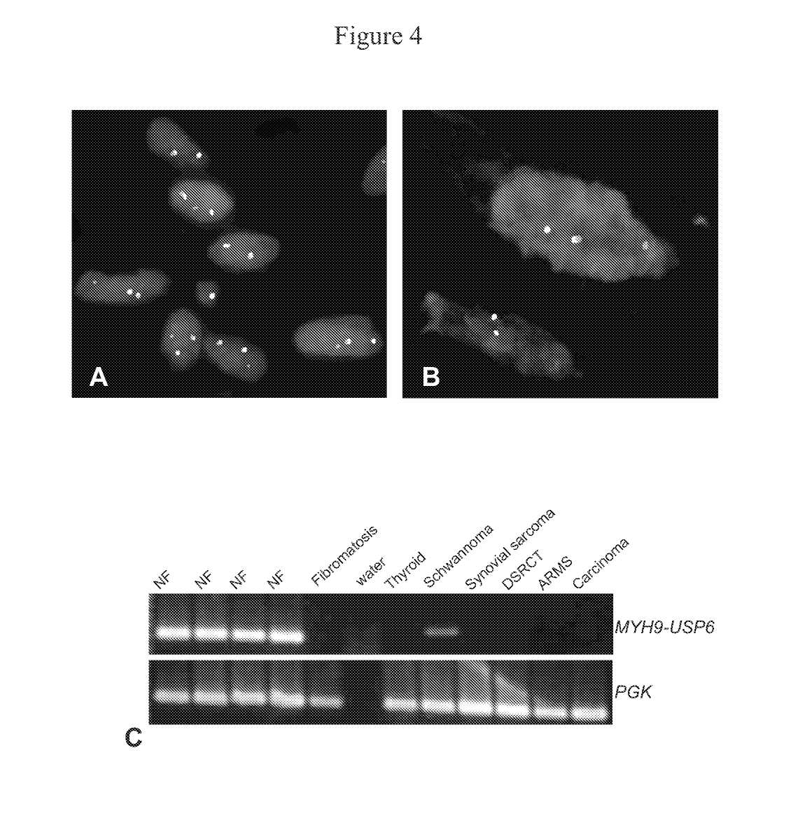

MYH9 3′ BACs included RP11-357J24, RP11-241E17, and RP11-877C15 (625 Kb), and MYH9 5′ BACs included RP11-643I13, RP11-744F17, and RP11-846O5 (896 Kb). A balanced rearrangement of MYH9 was found in 29/45 cases (64%) (Table 1 and FIG. 4A). To further confirm the fusion, a FISH bring together approach was taken. A bring together FISH probe was constructed by labeling the 3′ end of MYH9 in spectrum orange and the 5′ end of USP6 in spectrum green. DNA isolation, nick translation, and hybridization were performed as described elsewhere (Sukov et al., Skeletal Radiol., 37(4):321-327 (2008)). Tissues were scored by two independent investigators and considered positive if ≧10% of 200 cells showed split signals. FISH images were captured using a Leica DM 6000 scope (Leica, Bannockbun, Ill.) with Cytovision software (Genetix, Boston, Mass.). The FISH bring together approach showed that MYH9 was fused to USP6 (FIG. 4B). MYH9 was only rearranged when USP6 was rearranged. RT-PCR specific for the MYH9-USP6 fusion on two nodular fasciitis samples was performed as follows: RNA from formalin fixed paraffin embedded (FFPE) samples was extracted using 10 μm sections and extracted using established methods as described elsewhere (Qian et al., Diagn. Mol. Pathol., 14(1):23-28 (2005)). RT-PCR for MHY9-USP6 was performed on the extracted RNA from FFPE samples. One microgram RNA was reversed transcribed using iScript Select cDNA Synthesis Kit (BioRad, Hercules, Calif.) using random primers. PCR was performed using UPS6 primers:

| | |
|---|---|
| 5′ TACGATCGGCCTCCTGGGATG | (SEQ ID NO: 8) |
| and | |
| 5′ CTTCCGCTCCTGTGCCTGCAAACTA, | (SEQ ID NO: 9) |

MYH9 primers:

| | |
|---|---|
| 5′ ATCACCGCGGTTCCTG | (SEQ ID NO: 10) |
| and | |
| 5′ ACGGAAGGCTAAGCAAGGCTG, | (SEQ ID NO: 11) |

and cycling conditions: 94° C. 30 sec, 65° C. 30 sec, and 72° 30 sec) with a final extension of 72° C. for 10 min. Products were visualized on 3% gels and photographed using a DigiDoc-It Imager (UVP, Upland, Calif.). DNA was extracted using Qiaquick gel extraction columns (Qiagen, Valencia, Calif.) or ExoSap It (USB, Cleveland, Ohio) per manufacturer instructions. RT-PCR specific for the MYH9-USP6 fusion exhibited MYH9 noncoding exon 1 fused to part of USP6 noncoding exon 1 (Type I—GGGGCAGATCCAGGTTCAG—GAAACTGGGCATCTCTGTGGC; Genbank Accession No. 1385057; (SEQ ID NO:12)) and MYH9 noncoding exon 1 fused with USP6 coding exon 2 (Type II—GGGGCAGATCCAGGTTCAG—GATGGACATGGTAGAGAATGC; Genbank Accession No. 1394854; (SEQ ID NO:13)). The MYH9-USP6 fusion was further confirmed in 11/15 (73%) of FFPE nodular fasciitis cases and not in any controls (FIG. 4C).

Clinical cytogenetic and molecular characteristics of nodular fasciitis (n=45) are summarized in Table 1, and human nodular fasciitis histology is represented in FIG. 2A. Average age of nodular fasciitis patients was 32 years (range 4-70 years) with a 1:1.7 male to female ratio. Average lesion size was 1.7 cm (range=0.7-4.8 cm). 91% of human nodular fasciitis assayed contained rearrangement of the USP6 locus. The novel MYH9-USP6 fusion gene was found in most cases of human nodular fasciitis.

TABLE 1
Clinical, cytogenetic, and molecular features of nodular fasciitis
used in this study.

| | Sex* | | Size | USP6 (% | MHY9 (% | |
|---|---|---|---|---|---|---|
| ID | (Age)** | Location | (cm) | rearranged) | rearranged) | RT PCR |
| 1 | M(44) | palm | 1.6 | +(42) | +(27) | MYH9- |
| | | | | | | USP6(I &II) |
| 2 | F(56) | elbow | 1.7 | +(32) | +(40) | MYH9- |
| | | | | | | USP6(I &II) |
| 3 | M(30) | forehead | | +(67) | +(45) | MYH9- |
| | | | | | | USP6(I & II) |
| 4 | F(15) | thorax | | +(25) | +(27) | MYH9- |
| | | | | | | USP6(I & II) |
| 5 | F(33) | scapula | | +(61) | +(51) | MYH9-USP6(I) |
| 6 | M(37) | deltoid | 4.8 | +(25) | +(31) | MYH9-USP6(I) |
| 7 | F(53) | arm | | +(73) | +(68) | MYH9-USP6(I) |
| 8 | F(20) | ear | 0.8 | +(37) | +(29) | MYH9-USP6(I) |
| 9 | F(4) | parietal | | +(39) | +(41) | MYH9-USP6(I) |
| 10 | F(18) | orbital | 0.7 | +(42) | +(30) | MYH9-USP6(I) |
| 11 | F(41) | neck | 1.7 | +(66) | +(66) | MYH9-USP6(I) |
| 12 | M(49) | thigh | | +(24) | +(20) | — |
| 13 | F(18) | palm | 2.5 | +(43) | +(35) | — |
| 14 | M(36) | shoulder | 1.6 | +(35) | +(18) | — |
| 15 | M(53) | neck | | +(31) | +(45) | — |
| 16 | M(9) | back | | +(48) | +(35) | ND |
| 17 | F(47) | forearm | 1.0 | +(37) | +(33) | ND |
| 18 | M(50) | eyelid | | +(26) | +(20) | ND |
| 19 | M(29) | pectoral | | +(24) | +(33) | ND |
| 20 | M(36) | shoulder | | +(16) | +(18) | ND |
| 21 | F(54) | heel | | +(24) | +(17) | ND |
| 22 | F(9) | hand | | +(35) | +(31) | ND |
| 23 | M(13) | shoulder | 1.5 | +(41) | +(35) | ND |
| 24 | F(20) | ear | 1.8 | +(37) | +(18) | ND |
| 25 | M(29) | forehead | | +(48) | +(49) | ND |
| 26 | F(17) | knee | | +(25) | +(43) | ND |
| 27 | M(12) | upper | | +(62) | +(65) | |
| | | extremity | | | | |
| 28 | F(33) | forearm | | +(33)*** | +(12) | ND |
| 29 | F(33) | forearm | | +(42)*** | +(17) | ND |
| 30 | F(52) | neck | | +(12) | — | — |
| 31 | F(55) | back | | +(20) | — | — |
| 32 | F(20) | auricular | 0.8 | +(40) | — | — |
| 33 | F(21) | forearm | | +(39) | — | — |
| 34 | F(26) | forearm | 1.2 | +(55) | — | — |
| 35 | F(48) | submental | 2.8 | +(19) | — | — |
| 36 | M(31) | thigh | | +(23) | — | — |
| 37 | M(32) | forearm | | +(22) | — | ND |
| 38 | F(48) | forearm | | +(59) | — | ND |
| 39 | F(17) | chin | | +(49) | — | ND |
| 40 | F(19) | cervical | | +(43) | — | |
| | | mass | | | | ND |
| 41 | M(55) | lumbar | | +(55) | — | ND |
| 42 | F(70) | breast | | — | — | ND |
| 43 | F(35) | forearm | | — | — | ND |
| 44 | F(16) | back | | — | — | ND |
| 45 | M(22) | forearm | | — | — | — |

+, positive;
−, negative for rearrangement and/or RT-PCR.
*M = male,
F = female.
**Age in years.
*** amplified for USP6. FISH analysis and average scores of two independent investigators.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a mammal having a mesenchymal neoplasm as having nodular fasciitis, wherein said method comprises:
(a) determining whether or not a cell from said mesenchymal neoplasm contains a USP6 or MYH9 rearrangement, and
(b) classifying said mammal as having nodular fasciitis if said cell contains said rearrangement.

2. The method of claim 1, wherein said USP6 or MYH9 rearrangement is a translocation between USP6 and MYH9.

3. The method of claim 2, wherein said translocation results in the coding region of USP6 being under the control of a MYH9 promoter.

4. The method of claim 1, wherein said determining step comprises performing in situ hybridization.

5. The method of claim 1, wherein said determining step comprises performing in situ hybridization with a USP6 probe set.

6. The method of claim 1, wherein said determining step comprises performing in situ hybridization with a MYH9 probe set.

7. The method of claim 1, wherein said determining step comprises performing an assay to detect increased expression of a USP6 nucleic acid, wherein said increased expression indicates the presence of said rearrangement.

8. The method of claim 1, wherein said mammal is a human.

9. A method of identifying a mammal having a mesenchymal neoplasm as having nodular fasciitis, wherein said method comprises:
(a) detecting the presence of a USP6 or MYH9 rearrangement in a cell from said mesenchymal neoplasm, and
(b) classifying said mammal as having nodular fasciitis based at least in part on said presence of said rearrangement.

10. The method of claim 9, wherein said USP6 or MYH9 rearrangement is a translocation between USP6 and MYH9.

11. The method of claim 10, wherein said translocation results in the coding region of USP6 being under the control of a MYH9 promoter.

12. The method of claim 9, wherein said determining step comprises performing in situ hybridization.

13. The method of claim 9, wherein said determining step comprises performing in situ hybridization with a USP6 probe set.

14. The method of claim 9, wherein said determining step comprises performing in situ hybridization with a MYH9 probe set.

15. The method of claim 9, wherein said detecting step comprises detecting increased expression of a USP6 nucleic acid, wherein said increased expression indicates the presence of said rearrangement.

16. The method of claim 9, wherein said mammal is a human.

17. A probe set comprising at least one isolated nucleic acid molecule having the ability to hybridize to a USP6 nucleotide sequence and at least one isolated nucleic acid molecule having the ability to hybridize to a MYH9 nucleotide sequence.

18. The probe set of claim 17, wherein said isolated nucleic acid molecules are labeled.

19. The probe set of claim 17, wherein said at least one isolated nucleic acid molecule having the ability to hybridize to a USP6 nucleotide sequence comprises a label that is different from said label of said isolated nucleic molecule having the ability to hybridize to a MYH9 nucleotide sequence.

20. The probe set of claim 17, wherein said labels are fluorescent, chromogenic, or silver.
